(19) United States
(12) Patent Application Publication
(10) Pub. No.: US 2011/0257512 A1
(43) Pub. Date: Oct. 20, 2011

(54) SUSCEPTIBILITY-BASED LOCAL FLOW DETECTION TO CONTROL MR-GUIDED ABLATION USING BALLOON DEVICES

(75) Inventor: Sascha Krueger, Hamburg (DE)

(73) Assignee: KONINKLIJKE PHILIPS ELECTRONICS N.V., EINDHOVEN (NL)

(21) Appl. No.: 13/141,096

(22) PCT Filed: Nov. 23, 2009
(86) PCT No.: PCT/IB2009/055301

§ 371 Date: Jun. 21, 2011

Related U.S. Application Data
(60) Provisional application No. 61/141,956, filed on Dec. 31, 2008.

(51) Int. Cl. A61B 5/05; A61N 7/00

(52) U.S. Cl. 600/411; 601/2

(57) ABSTRACT

An interventional instrument (24) for use in performing an interventional procedure includes: a balloon (30) disposed proximate to a tip of the interventional instrument that inflates and anchors in a lumen of a fluid conduit during the interventional procedure; and one or more susceptibility markers (34) disposed proximate to the tip of the interventional instrument. A magnetic resonance scanner (10) is configured to image at least the tip of the interventional instrument during the interventional procedure using a magnetic resonance imaging sequence in which fluid flow (40) through the fluid conduit past the inflated balloon produces an extended magnetic resonance image artifact (42).

The following relates to the medical arts, cardiovascular medicine arts, magnetic resonance arts, interventional magnetic resonance arts, and related applications.

Interventional procedures employing catheters or other interventional instruments are known. The interventional instrument is inserted into an artery or vein or other fluid conduit in a human or animal subject, and is manipulated under visual guidance, e.g. provided by magnetic resonance imaging or another medical imaging modality, such that a tip of the interventional instrument is moved proximate to a cardiac vessel, cardiac valve, cardiovascular musculature, or other anatomical feature or region to undergo interventional therapy. In such procedures, it is often desired to anchor the tip of the interventional instrument at the desired position. In a balloon catheter, a small balloon disposed at the tip is deflated during insertion, and is then inflated when the tip reaches the desired position such that the inflated balloon expands, presses against the vessel lumen, and secures the distal part of the catheter. In balloon angioplasty, inflation of the balloon is intended to mechanically widen a vessel constriction or stenosis. In some other procedures, the inflated balloon anchors the tip of the interventional instrument to provide a stationary reference for performing another therapeutic procedure, e.g. pulmonary vein isolation for treatment of atrial fibrillation using tissue ablation. In this case, the balloon is part of the ablation catheter and, during treatment, is typically seated in one of the ostiae of a pulmonary vein.

In this illustrative interventional procedure of intravascular tissue ablation employing a balloon catheter, an RF electrode or a transducer such as an ultrasound transducer, semiconductor laser device, cryogenic device, or so forth outputs energy that locally disrupts proximate tissue in order to block arrhythmic signals or provide other therapy. In this technique, the anchor provided by the catheter tip balloon provides a well-defined reference position for the ablation therapy. The technique is used, for example, to treat cardiac arrhythmias such as atrial fibrillation.

Interventional procedures employing a catheter or other interventional instrument call for accurate control over the anchoring of the catheter tip via the balloon. For example, in atrial fibrillation ablation of the pulmonary veins, balloon multi-point ablation has the advantage of simplifying the positioning of the ablation device, speeding up the procedure of a full circumferential ablation and also ensuring completeness of the pulmonary vein isolation (PVI). Typically radio-frequency (RF), ultrasound, cryogenic (i.e., cryo), or laser ablation can be performed using such balloon devices. For some such procedures, a uniform contact to the tissue is desired so as to ensure a closed ablation ring.

Problematically, the magnetic resonance imaging typically used to guide insertion of the interventional instrument into the subject has not heretofore been useful for verifying and monitoring the anchoring of the balloon in the vein or other fluid conduit. Instead, x-ray guidance is typically used to monitor the balloon tip anchoring. In this approach, the balloon anchoring and sealing of the fluid conduit is monitored using contrast-enhanced fluoroscopy to assess the residual pulmonary vein flow passing the positioned and inflated balloon. In case of cryoablation, this method detects whether cryoadherence is effective to stabilize the device, and monitors the seal to ensure persistent proper tissue contact during an RF ablation. The use of x-ray guidance has certain disadvantages, including the use of ionizing x-radiation, and the use of nephrotoxic contrast agents or other invasive contrast agent media.

The following provides new and improved apparatuses and methods which overcome the above-referenced problems and others.

In accordance with one disclosed aspect, an interventional instrument comprises: an elongate portion configured for insertion into a fluid conduit of a human or animal subject; a balloon disposed proximate to a tip of the elongate portion and sized to anchor in a lumen of the fluid conduit when inflated; and one or more susceptibility markers disposed proximate to the tip of the elongate portion and configured to generate an extended magnetic resonance image artifact corresponding to fluid flow through the fluid conduit past the inflated balloon.

In accordance with another disclosed aspect, an interventional system comprises: an interventional instrument for use in performing an interventional procedure, the interventional instrument including (i) a balloon disposed proximate to a tip of the interventional instrument that inflates and anchors in a lumen of a fluid conduit during the interventional procedure and (ii) one or more susceptibility markers disposed proximate to the tip of the interventional instrument; and a magnetic resonance scanner configured to image at least the tip of the interventional instrument during the interventional procedure using a magnetic resonance imaging sequence in which fluid flow through the fluid conduit past the inflated balloon produces an extended magnetic resonance image artifact.

In accordance with another disclosed aspect, an interventional procedure comprises: inserting a tip of an interventional instrument into a fluid conduit of a human or animal subject; inflating a balloon proximate to the tip of the interventional instrument to block fluid flow through the fluid conduit, the inflated balloon comprising a paramagnetic or ferromagnetic material; and assessing the blockage of fluid flow by performing magnetic resonance imaging of the inflated balloon comprising the paramagnetic or ferromagnetic material.

One advantage resides in elimination of administration of an intravascular contrast agent during interventional therapy employing a balloon catheter.

Another advantage resides in avoiding the use of ionizing radiation during monitoring of balloon anchoring and vascular seal quality during an interventional procedure employing a balloon catheter.

Another advantage resides in enabling the use of a magnetic resonance scanner for monitoring of balloon anchoring and vascular seal quality during an interventional procedure employing a balloon catheter e.g. to ensure proper full circumferential contact during ablation of the pulmonary veins in the treatment of atrial fibrillation.

Further advantages will be apparent to those of ordinary skill in the art upon reading and understand the following detailed description.

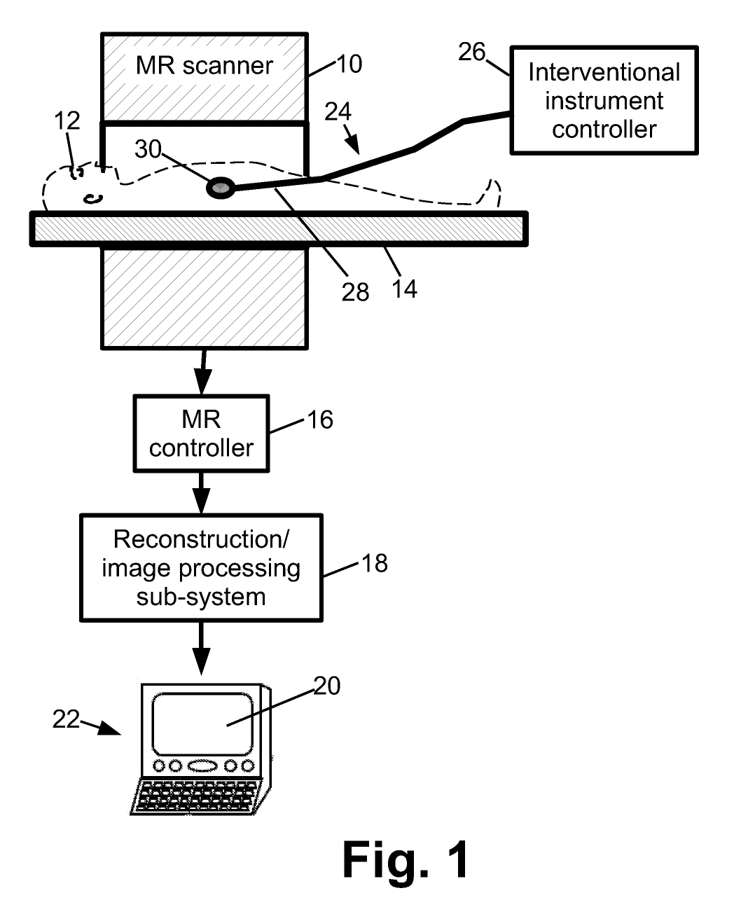

FIG. 1 diagrammatically shows an interventional magnetic resonance system including an interventional instrument having a balloon-equipped tip in which the balloon includes at least one susceptibility marker, and a magnetic resonance scanner for monitoring the interventional procedure.

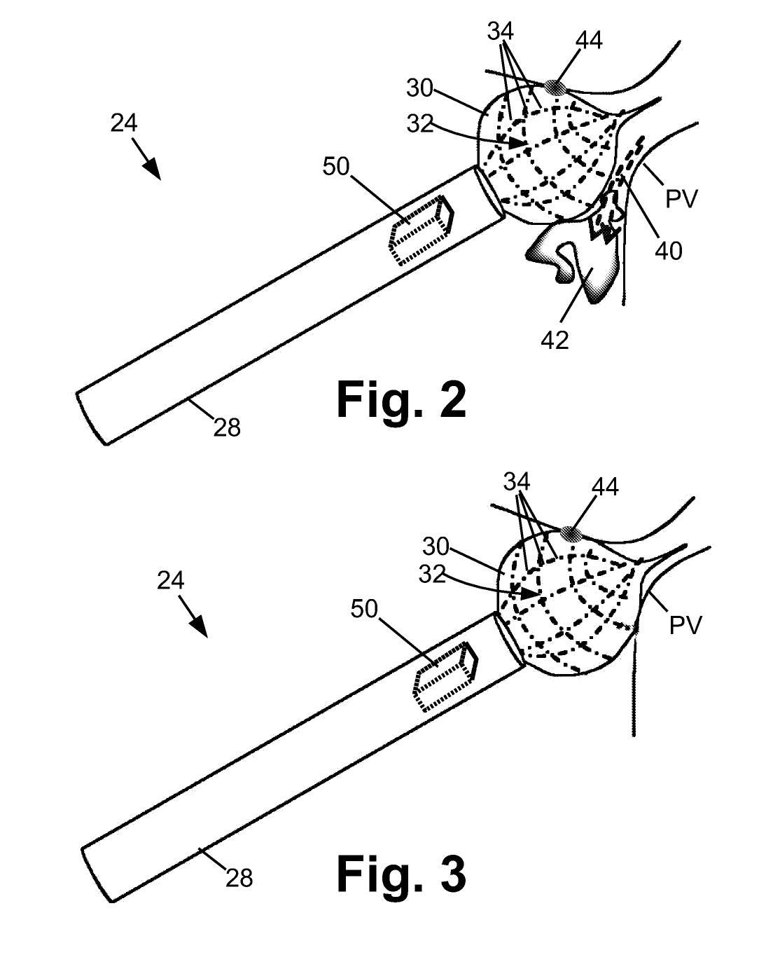

FIG. 2 diagrammatically shows a schematic representation of the catheter tip with the balloon in non-uniform contact with the vessel lumen resulting in a non-localized image artifact due to flowing dephased spins.

FIG. 3 diagrammatically shows a schematic representation of the catheter tip with the balloon in substantially uniform contact with the vessel lumen.

With reference to FIG. 1, an interventional magnetic resonance system includes a magnetic resonance (MR) scanner 10 that images a human subject 12 undergoing an interventional procedure. The subject 12 is disposed on a suitable subject support 14. Instead of a human subject, an animal subject undergoing a veterinary or preclinical interventional procedure is also contemplated. The MR scanner 10 may be, for example, an Achieva™, Intera™, or Panorama™ MR system (available from Koninklijke Philips N. V., Eindhoven, the Netherlands), although other commercial or non-commercial

MR scanners can also be used. The illustrated MR scanner 10 is a horizontal cylindrical bore scanner shown diagrammatically in side-sectional view to review the subject 12 inside the scanner bore; however, an open bore scanner such as the Panorama™ MR system can also be used. An open MR scanner may provide an advantage in terms of increased freedom of movement for the physician or other medical personnel or robotic apparatus performing the interventional procedure.

The MR scanner 10 is controlled by suitable power, electronics, and other control components for powering a resistive or superconducting main magnet to generate a static (B₀) magnetic field, for driving magnetic field gradient coils to superimpose selected magnetic field gradients on the static B₀ magnetic field, for energizing a radio frequency coil or coil array to generate radio frequency (B₁) fields for exciting magnetic resonance, for receiving magnetic resonance signals via a radio frequency coil or coil array (which in general may be the same or a different coil or coil array than that used for the B₁ field generation), and so forth. These power, electronics, and other components are collectively diagrammatically indicated in FIG. 1 as an MR controller 16. The MR scanner 10 and scan controller 16 can be used to implement substantially any magnetic resonance imaging sequence, such as echo planar imaging (EPI), steady-state free precession (SSFP) imaging, or so forth. The resulting imaging data are suitably processed by an image reconstruction and image processing subsystem 18, which may be embodied by a suitably programmed computer or other programmable digital device, or by application-specific integrated circuitry (ASIC), or by a combination of a programmed digital processor and ASIC, or so forth. The image reconstruction and image processing subsystem 18 employs Fourier transform image reconstruction or another image reconstruction algorithm comporting with the spatial encoding implemented by the imaging sequence so as to generate a reconstructed image from the acquired magnetic resonance imaging data. The reconstructed image, or a selected portion thereof such as a two-dimensional slice or a maximum intensity projection (MIP) or so forth, is suitably displayed on the display 20 of a computer 22 or other user interface device. In some embodiments, the user interface computer 22 also embodies some or all of the MR controller 16, and/or some or all of the reconstruction or image processing components 18.

With continuing reference to FIG. 1 and with further reference to FIGS. 2 and 3, an interventional procedure is performed using an interventional instrument 24 operating in conjunction with suitable control electronics, power delivery components, robotic manipulators, or so forth that are collectively represented in FIG. 1 by an interventional instrument controller 26. The interventional instrument 24 includes an elongate portion 28 at least the tip of which is configured for insertion into blood vessel or other fluid conduit of a human or animal subject, and a balloon 30 disposed proximate to the tip of the elongate portion 28. It is to be appreciated that the drawings are not to scale, and in particular the balloon 30 is drawn substantially larger than a typical actual size in FIG. 1. The drawings also illustrate the balloon 30 in an inflated configuration; in general, the balloon is deflated during insertion of the elongate portion 28 into the subject 12. The elongate portion 28 and the balloon 30 may, for example, define a balloon catheter. During insertion, the magnetic resonance scanner 10 is used to monitor the position of the elongate portion 28, and in particular its tip, inside the subject 12. In an illustrative cardiac procedure, the elongate portion 28 is inserted in such a way and until the tip is located in a lumen of a blood vessel in or near the heart. Once the elongate portion 28 is located in a desired position, for example near a pulmonary vein ostium PV in the case of an illustrative pulmonary vein isolation (PVI) procedure, the balloon 30 is inflated to engage with the lumen of the pulmonary vein PV so as to anchor the balloon 30, and hence the tip of the elongate portion 28, in a fixed reference position. More generally, the balloon 30 is sized to anchor in a lumen of the fluid conduit when inflated. It will be appreciated that this sizing need not be particularly precise, since the inflating balloon can expand over a substantial range to engage with the lumen walls. Inflation of the balloon 30 is achieved using a suitable fluid inlet pathway (not shown) passing from the controller 26 through the elongate portion 28 and connecting with the balloon 30. In some embodiments, a water-based fluid 32 fills the inflated balloon 30.

The balloon 30 proximate to the tip of the interventional instrument 24 inflates to block fluid flow through the fluid conduit, specifically blood flow in the pulmonary vein PV in the illustrated case. However, the blockage of fluid flow may be incomplete if the balloon 30 seats in an unsatisfactory fashion in the venous lumen.

To address this problem, the inflated balloon 30 comprises a paramagnetic or ferromagnetic material that defines one or more susceptibility markers. Some examples of suitable ferromagnetic materials include: steel, iron, nickel, cobalt, gadolinium, or dysprosium. Some examples of suitable paramagnetic (encompassing super-paramagnetic) materials include: iron-dioxide, dysprosium-dioxide, and chromium-dioxide. In the illustrated embodiment, the paramagnetic or ferromagnetic material takes the form of a matrix of susceptibility markers 34 disposed on the inflated balloon. In other contemplated embodiments, the paramagnetic or ferromagnetic material may take the form of paramagnetic or ferromagnetic particles impregnating the balloon. In yet other contemplated embodiments in which the balloon 30 is inflated using the illustrated water-based fluid 32, it is contemplated for the paramagnetic or ferromagnetic material to comprise paramagnetic or ferromagnetic particles suspended in the water-based fluid 32 in the inflated balloon 30. Various combinations of these arrangements are also contemplated.

The susceptibility markers 34 enable assessment of the blockage of fluid flow by the inflated balloon 30 by performing magnetic resonance imaging of the inflated balloon 30 comprising a paramagnetic or ferromagnetic material 34. Local blood flow 40 around the inflated balloon 30 is detectable in the magnetic resonance image due to susceptibility-tagging of the protons of the blood flow 40 past the paramagnetic or ferromagnetic material-marked inflated balloon 30. Protons of the blood flow 40 past the inflated balloon 30 in close proximity to the paramagnetic or ferromagnetic material 34 accumulate additional phase and are thus visualized in magnetic resonance images as an extended magnetic resonance image artifact 42 (see FIG. 2) corresponding to the fluid flow (venous blood flow 40, in FIG. 2) through the fluid conduit (the pulmonary vein ostiae PV in FIG. 2) past the unsatisfactory seal provided by the inflated balloon 30 in FIG. 2. Substantially any magnetic resonance imaging technique, including for example echo planar imaging (EPI) or balanced steady state free precession sequence (balanced SSFP) imaging, can be used. Advantageously, the dephased blood defining the image artifact 42 is extended in space in case of non-perfect positioning of the device resulting in local residual flow, and therefore the extended image artifact 42 is readily distinguished from discrete susceptibility artifacts due to stray ferromagnetic particles or the like (such as an illustrated discrete susceptibility artifact 44 in case of perfect seal and absence of local flow) by the extended nature or distribution of the flow-related image artifact 42. Therefore local flow can be measured at the balloon 30 by local phase-tagging of protons by the paramagnetic or ferromagnetic material 34.

Detection of the local blood flow 40 enables assessment in real time of the sealing against fluid flow provided by the inflated balloon 30. FIG. 3 depicts the situation in which the inflated balloon 30 provides a satisfactory seal against venous blood flow. The extended susceptibility artifact 42 is not present, because the blood flow 40 is eliminated by the satisfactory seal. The discrete susceptibility artifact 44 remains visible in FIG. 3, but is readily recognized as unrelated to blood flow due to the discrete and non-extended nature of the artifact 44.

The disclosed approach for assessing the seal provided by the inflated balloon 30 can be performed using regular real-time magnetic resonance imaging sequences such as EPI or SSFP. The use of cinematic (CINE) image sequences is sometimes useful for detecting the extended susceptibility artifact 42. Advantageously, magnetic resonance imaging is typically used to guide insertion of the interventional instrument 24 into the subject 12. Thus, the disclosed approach enables a single imaging modality, namely magnetic resonance imaging, to provide both guidance for inserting the interventional instrument 24, and assessment of the seal against fluid flow provided by the inflated balloon 30. As a further advantage, the marker matrix 34 provides accurate localization of the inflated balloon 30 with respect to the pulmonary vein ostiae PV. Magnetic resonance imaging moreover offers the advantage of soft tissue contrast and vessel lumen visualization without requiring additional contrast agents, making for the excellent guidance capabilities of magnetic resonance imaging.

The interventional procedure is performed once the balloon seal is verified. For a balloon angioplasty procedure, the balloon inflation continues so as to exert pressure on the vessel lumen walls to mechanically widen a vessel constriction or stenosis. The disclosed technique for monitoring the anchoring and sealing provided by the balloon 30 can be used throughout the angioplasty procedure to ensure that the balloon 30 does not shift in position, develop a blood flow leak, or otherwise fail.

With continuing reference to FIGS. 2 and 3, for a tissue ablation procedure a suitable electrode or transducer 50 is disposed proximate to the tip of the elongate portion and configured to emit energy that ablates proximate tissue of the subject. For example, the element 50 may be an electrode comprising a conductive element providing contact to the tissue for delivering energy to the tissue. The element 50 may alternatively be a transducer 50 such as, for example: a semiconductor laser that converts electrical energy into optical energy to perform laser ablation; a radio frequency (RF) microtransmitter that converts electrical energy to RF output to perform ablation; an ultrasonic transducer that converts electrical energy into ultrasonic energy that ablates the tissue; or so forth. The transducer 50 is suitably powered by electrical wires (not shown) passing through the elongated portion 28 of the interventional instrument 24. If the balloon 30 is configured to inflate by filling with the illustrated water-based fluid 32, then in some embodiments the transducer 50 is an ultrasonic transducer configured to emit ultrasonic energy focused into tissue proximal to the balloon to ablate respective tissue of the subject 12. For laser ablation, the transducer 50 is optionally replaced by an optical fiber running with the elongate portion 28 so as to carry laser energy from an external source to the tip of the elongate portion 28.

The disclosed techniques for assessment of the seal against fluid flow can be used in MR-guided electrophysiology interventions involving circumferential ablation of pulmonary veins using a balloon 30 inflated using water-based fluid 32. The technique is also applicable for other types of intravascular flow measurements, or to assess the local convective heat transport during transcutaneous MR-guided tumor ablation. For cardiac interventional procedures generally, the balloon 30 is configured to anchor in a lumen of a blood vessel in or near the heart when inflated.

If the magnetic resonance imaging sequence used for assessing the balloon seal employs a long echo time, then the imaging sequence optionally can also be used to assess temperature. The proton resonance frequency is a function of temperature, and has about a 100 part-per-million (ppm) frequency shift per degree centigrade (° C.). That is, the proton resonance frequency shifts with temperature at a rate of about 100 ppm/° C. Thus, temperature mapping based on a temperature shift of magnetic resonance signals can be performed in the vicinity of the tissue ablation procedure continously and simultaneously with the assessment of the balloon seal.

Still further, while the illustrated susceptibility markers 34 are shown disposed on the inflated balloon 30, the suspectibility marker or markers may also be disposed elsewhere proximate to the tip of the interventional instrument 24, such as near the distal end of the elongate portion 28 proximate to the tip of the interventional instrument 24.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An interventional instrument comprising:
an elongate portion configured for insertion into a fluid conduit of a human or animal subject;
a balloon disposed proximate to a tip of the elongate portion and sized to anchor in a lumen of the fluid conduit when inflated; and
one or more susceptibility markers disposed proximate to the tip of the elongate portion and configured to generate an extended magnetic resonance image artifact corresponding to fluid flow through the fluid conduit past the inflated balloon.

2. The interventional instrument as set forth in claim 1, wherein the one or more susceptibility markers are disposed on or in the balloon.

3. The interventional instrument as set forth in claim 1, wherein the elongate portion and the balloon define a balloon catheter.

4. The interventional instrument as set forth in claim 1, wherein the elongate portion is configured for insertion into a vascular system of the subject, and the balloon is sized to anchor in a lumen of a blood vessel in or near the heart when inflated.

5. The interventional instrument as set forth in claim 1, further comprising:
an electrode or transducer disposed proximate to the tip of the elongate portion and configured to emit energy that ablates proximate tissue of the subject.

6. The interventional instrument as set forth in claim 5, wherein the balloon is configured to inflate by filling with a water-based fluid, and the transducer comprises an ultrasonic transducer configured to emit ultrasonic energy focused into tissue proximal to the balloon to ablate respective tissue of the subject.

7. The interventional instrument as set forth in claim 6, wherein the one or more susceptibility markers comprise:
paramagnetic or ferromagnetic particles suspended in the water-based fluid in the inflated balloon.

8. The interventional instrument as set forth in claim 1, wherein the one or more susceptibility markers disposed on or in the balloon comprise:
a matrix of susceptibility markers disposed on the inflated balloon.

9. The interventional instrument as set forth in claim 1, wherein the one or more susceptibility markers comprise a paramagnetic or ferromagnetic material.

10. The interventional instrument as set forth in claim 1, wherein the one or more susceptibility markers comprise paramagnetic or ferromagnetic particles impregnating the balloon.

11. The interventional instrument as set forth in claim 1, wherein the one or more susceptibility markers comprise a material selected from the group consisting of: steel, iron, nickel, cobalt, gadolinium, dysprosium, iron-dioxide, dysprosium-dioxide, and chromium-dioxide.

12. An interventional system comprising:
an interventional instrument for use in performing an interventional procedure, the interventional instrument including (i) a balloon disposed proximate to a tip of the interventional instrument that inflates and anchors in a lumen of a fluid conduit during the interventional procedure and (ii) one or more susceptibility markers disposed proximate to the tip of the interventional instrument; and
a magnetic resonance scanner configured to image at least the tip of the interventional instrument during the interventional procedure using a magnetic resonance imaging sequence in which fluid flow through the fluid conduit past the inflated balloon produces an extended magnetic resonance image artifact.

13. The interventional system as set forth in claim 12, wherein the magnetic resonance scanner is configured to image at least the tip of the interventional instrument during the interventional procedure using one of (i) a steady state free precession (SSFP) imaging sequence and (ii) an echo planar imaging (EPI) sequence.

14. The interventional system as set forth in claim 12, wherein the magnetic resonance scanner is configured to image at least the tip of the interventional instrument during the interventional procedure using cinematic (CINE) imaging.

15. The interventional system as set forth in claim 12, wherein the interventional instrument is configured to perform a tissue ablation procedure.

16. The interventional system as set forth in claim 12, wherein the magnetic resonance scanner is further configured to perform temperature mapping based on a temperature shift of magnetic resonance signals.

17. An interventional procedure comprising:
inserting a tip of an interventional instrument into a fluid conduit of a human or animal subject;
inflating a balloon proximate to the tip of the interventional instrument to block fluid flow through the fluid conduit, the inflated balloon comprising a paramagnetic or ferromagnetic material; and
assessing the blockage of fluid flow by performing magnetic resonance imaging of the inflated balloon comprising the paramagnetic or ferromagnetic material.

18. The interventional procedure as set forth in claim 17, further comprising:
determining a satisfactory placement of the inflated balloon based on the assessing; and
performing a therapeutic procedure subsequent to the determining.

19. The interventional procedure as set forth in claim 18, wherein the therapeutic procedure comprises a tissue ablation procedure.
